(19) United States
(12) Patent Application Publication
(10) Pub. No.: US 2010/0311036 A1
(43) Pub. Date: Dec. 9, 2010

(54) Methods for Augmentation of Cell Cryopreservation

(75) Inventor: Xiaoming He, Lexington, SC (US)

Correspondence Address: DORITY & MANNING, P.A., POST OFFICE BOX 1449, GREENVILLE, SC, 29602-1449, US

(73) Assignee: UNIVERSITY OF SOUTH CAROLINA, Columbia, SC (US)

(21) Appl. No.: 12/796,994

(22) Filed: Jun. 9, 2010

Related U.S. Application Data
(60) Provisional application No. 61/268,118, filed on Jun. 9, 2009.

(51) Int. Cl. C12N 5/02

(52) U.S. Cl. 435/2; 435/374; 977/923

(57) ABSTRACT

In accordance with certain embodiments of the present disclosure, a method for cryopreserving a cell is described. The method includes encapsulating a cell in a microcapsule, the microcapsule having a diameter of less than about 100 μM. The method further includes vitrifying the encapsulated cell in a vitrifying solution comprising a cryoprotectant, wherein the cell is cooled at a rate of equal to or greater than 30,000° C./min and the cryoprotectant is present at a concentration of less than or equal to 1.5 M.

### CROSS-REFERENCE TO RELATED APPLICATION

The present application is based on and claims priority to U.S. Provisional Application 61/268,118 having a filing date of Jun. 9, 2009, which is incorporated by reference herein.

### BACKGROUND

Both cell microencapsulation and cryopreservation are critical to the eventual success of modern cell based medicine. With recent advances in tissue engineering, regenerative medicine, cell transplantation, stem cell therapy, and assisted reproduction, the living cell is becoming increasingly important as a therapeutic tool in modern medicine. A major challenge to the eventual success of these emerging cell-based medical technologies is the limited availability of the desired cell sources, which can be alleviated by cell microencapsulation and cryopreservation. The former allows the use of non-autologous cells for the treatment of human diseases by avoiding undesired immune response (i.e., immunoisolation), which is achieved by encapsulating the cells in microcapsules with the appropriate permeability to exclude immunocytes, antibodies and complement factors generated by the host immune system while allowing controlled and sustained release of therapeutic products produced by the cells. On the other hand, cell cryopreservation makes it possible to establish cell banks of important living cells for wide distribution to end users so that they are readily available when needed in the future. Moreover, long-term stabilization of important living cells by cryopreservation is much cheaper and less time-consuming than maintaining the cells in culture.

Microcapsules made of alginate, a natural biodegradable and biocompatible polysaccharide, have been widely used to encapsulate a variety of sensitive mammalian cells including embryonic stem cells. However, alginate microcapsules used for encapsulation of embryonic stem cells reported in the literature are larger than 300 μm but smaller microcapsules are desired for better efficacy. Recently, C3H10T1/2 mouse mesenchymal stem cells have been successfully encapsulated in much smaller (˜100 μm) alginate microcapsules. Considering that clinical practice of microencapsulation technology has been limited by the sites for transplantation of large microcapsules and inadequate transport of oxygen, nutrients, metabolites to and from microencapsulated cells in large microcapsules, it is desired to use microcapsules as small as possible. For a sphere, the specific surface area (the ratio of surface area to volume) increases as the diameter of the microcapsule decreases. Therefore, a decrease in microcapsule diameter should facilitate the transport of oxygen and nutrients to the encapsulated cells (particularly those in the center) and enhance the release of therapeutic molecules secreted by the cells out of the microcapsule. Moreover, small microcapsules have been shown to have better mechanical property/stability and biocompatibility. A reduction in diameter also allows more choices of implantation sites. Last but not the least, small microcapsules are easier to cryopreserve for long term storage than large ones.

Cell cryopreservation by slowing freezing and conventional vitrification is sub-optimal. Currently, the two most commonly used approaches for cryopreservation of mammalian cells are slow-freezing (i.e., with ice formation and cooling rates generally less than 100° C. per minute) and the conventional vitrification (i.e., without ice formation). A summary of the advantages and disadvantages of the two approaches is given in FIG. 1. Although a low, non-toxic concentration of cryoprotective agent (CPA) (low-CPA, generally ≦1.5 M) is used for the conventional slow-freezing approach, it is always associated with cell injury due to ice formation, freeze concentration of solutes, and prolonged exposure to cryoprotectants and chilling temperatures. Cryopreservation by vitrification avoids ice formation altogether and has been proven to be a promising alternative to slow-freezing for cryopreserving sensitive mammalian cells. The cryoprotectant concentration required for the conventional vitrification approach at a slow cooling rate (i.e., less than 1000° C. per minute), however, is very high (e.g., >7 M when glycerol is used as the cryoprotectant). Therefore, various techniques have been used to reduce the cryoprotectant concentration required for vitrification by creating high cooling rates (usually <20,000° C./min). These techniques include electron microscope grid, open pulled straw, solid surface at a cryogenic temperature, microliter (μl) sized droplets, cryotop, nylon mesh, cryotip, and cryoloops. As a result, the cryoprotectant concentration required for vitrifying important mammalian cells such as the embryonic stem cells can be reduced to as low as 4 M, which unfortunately still can cause significant osmotic and metabolic cell injury in an exposure time as short as minutes. Consequently, it is necessary to use multiple steps of cryoprotectant loading/dilution and maintain short exposure time to the high concentration of cryoprotectants in each step to minimize injury, which make the vitrification procedure complicated, stressful, and difficult to control. Therefore, it is of great interest and importance to reduce the cryoprotectant concentration required for vitrification to a low, non-toxic level such as that used in slow freezing.

Therefore, a need exists for a method of encapsulating cells in small microcapsules and effectively cryopreserving the microencapsulated cells by low-CPA vitrification (i.e., vitrification at a low, non-toxic concentration of cryoprotectant, ≦1.5 M) to overcome the shortcomings of conventional methods.

### SUMMARY

In accordance with certain embodiments of the present disclosure, a method for cryopreserving a cell is described. The method includes encapsulating a cell in a microcapsule, the microcapsule having a diameter of less than about 100 μm. The method further includes vitrifying the encapsulated cell in a vitrifying solution comprising a cryoprotectant, wherein the cell is cooled at a rate of equal to or greater than 30,000° C./min and the cryoprotectant is present at a concentration of less than or equal to 1.5 M.

### BRIEF DESCRIPTION OF THE FIGURES

A full and enabling disclosure of the present subject matter, including the best mode thereof, to one of ordinary skill in the art, is set forth more particularly in the remainder of the specification, including reference to the accompanying figures in which:

FIG. 1 illustrates a summary of the major advantages and disadvantages of the conventional slow freezing and vitrification approaches for single cell cryopreservation: the proposed low-CPA vitrification technique combines the advantages of the two conventional approaches while avoiding their shortcomings.

FIG. 2 illustrates a comparison of the quartz microcapillary and the conventional French type plastic straw for cell vitrification showing the difference in size of the two devices.

FIG. 3 illustrates a schematic of the electrostatic spray device for preparing small (˜100 μm) alginate microcapsules.

FIG. 4 illustrates typical micrographs of microcapsules with good (round in shape and uniform in size, A) and bad (irregular in shape and non-uniform in size, B) morphology: Scale bar, 200 μm.

FIG. 5 illustrates data showing the effect on microcapsule diameter of sodium alginate concentration (A), spray voltage (B), and spray flow rate (C).

FIG. 6 illustrates a phase contrast image of microencapsulated cells (A) and fluorescent image of live (green) and dead (red) cells (B), scale bar: 100 μm.

FIG. 7 illustrates viability and proliferation/growth of fresh (control) and cryopreserved murine embryonic stem (ES) cells: (A), immediate and 1 day viability assessed by membrane integrity and attachment efficiency, respectively; and (B), proliferation/growth of control cells and cells cryopreserved by ultrafast vitrification with 2 M PROH and 0.5 M trehalose in three days. Trehalose (0.5 M) is present only in the extracellular medium while PROH exists both inside and outside the cells. PROH: 1,2-propanediol.

FIG. 8 illustrates micrographs showing the undifferentiated properties of mouse ES cells post vitrification: (A) staining for the surface glycoprotein SSEA-1; (B) GFP expression denoting transcriptional activity; (C) merged view of SSEA-1, GFP and nuclei staining; (D) phase contrast image of the two colonies; and alkaline phosphatase expression viewed by 10× (E) and 4× (F) objectives.

FIG. 9 illustrates a schematic of the method for plunging the quartz micro-capillary loaded with microencapsulated ES cells into liquid nitrogen (LN2).

FIG. 10 illustrates microcapsules before cryopreservation in 1× saline (A) and post cryopreservation in 1× saline with 0 (B), 5 (C), and 10% (v/v) (D) DMSO: Scale bars, 100 μm.

FIG. 11 illustrates calorimetric data showing the heat flux peak of ice melting for samples with (solid line) and without (dash line) microcapsules in the presence of 0 (A), 5 (B), 10 (C), and 15% (v/v) (D) DMSO.

FIG. 12 illustrates fractions of ice formation (F_{ice} in Eq. 1) in samples with and without microcapsules and their ratios in the presence of 0-15% (v/v): Error bars represent standard deviation.

FIG. 13 illustrates microcapsules before (A) and after (B) cryopreservation in 1× saline with 0.15 M Ca²⁺ (no DMSO) and microcapsules post cryopreservation in 1× saline with 7.5% (v/v) DMSO and 0 (C), 0.05 (D), 0.1 (E), and 0.15 M (F) Ca²⁺: Scale bars, 100 μm.

FIG. 14 illustrates fractions of ice formation (F_{ice}in Eq. 1) in samples with and without microcapsules and their ratios in the presence of 7.5% (v/v) and 0-0.15 M Ca²⁺: Error bars represent standard deviation.

FIG. 15 illustrates typical phase (A, C, E, and G) and fluorescent (B, D, F, and H) micrographs of control (non-microencapsulated, A-D) and microencapsulated (E-H)C3H10T1/2 mouse mesenchymal stem cells: Panels A and B are for control cells before cryopreservation, C and D are for control cells after cryopreservation, E and F are for microencapsulated cells before cryopreservation, and G and H are for microencapsulated cells after cryopreservation by vitrification using a 400 μm quartz microcapillary and 1.4 M DMSO. In the fluorescent micrographs, live and dead cells were stained green and red, respectively. Scale bars: 100 μm.

FIG. 16 illustrates proliferation of the encapsulated and non-microencapsulated C3H10T1/2 mouse mesenchymal stem cells after vitrification shown in FIG. 15 together with control (fresh without vitrification) 3 days after seeding them in 96 well plate and culturing in normal medium at 37° C. and 5% CO₂. The microencapsulated cells after vitrification were released from the microcapsule by ion-chelating using 0.055 M sodium citrate solution for 1 minute before seeding. Clearly, much more microencapsulated than non-microencapsulated cells after vitrification were able to attach at day 1 and the attached cells were able to proliferate normally just like the control (fresh) cells without vitrification.

### DETAILED DESCRIPTION

Reference will now be made in detail to embodiments of the disclosed subject matter, one or more examples of which are set forth below. Each example is provided by way of explanation of the subject matter, not limitation of the subject matter. In fact, it will be apparent to those skilled in the art that various modifications and variations can be made in the present disclosure without departing from the scope or spirit of the subject matter. For instance, features illustrated or described as part of one embodiment, can be used on another embodiment to yield a still further embodiment.

The methods described herein will have a long-lasting impact on advancing the fields of tissue engineering, regenerative medicine, cell/organ transplantation, stem cell therapy, and assisted reproduction, because all these emerging medical technologies require the wide distribution of important living cells to end users for future use. Utilizing the methodology described herein will provide the foundation to satisfy this requirement of the emerging cell-based medicine by encapsulating important living cells such as ES cells in small microcapsules and effectively cryopreserve the microencapsulated cells by low-CPA vitrification for wide distribution to end users and future use.

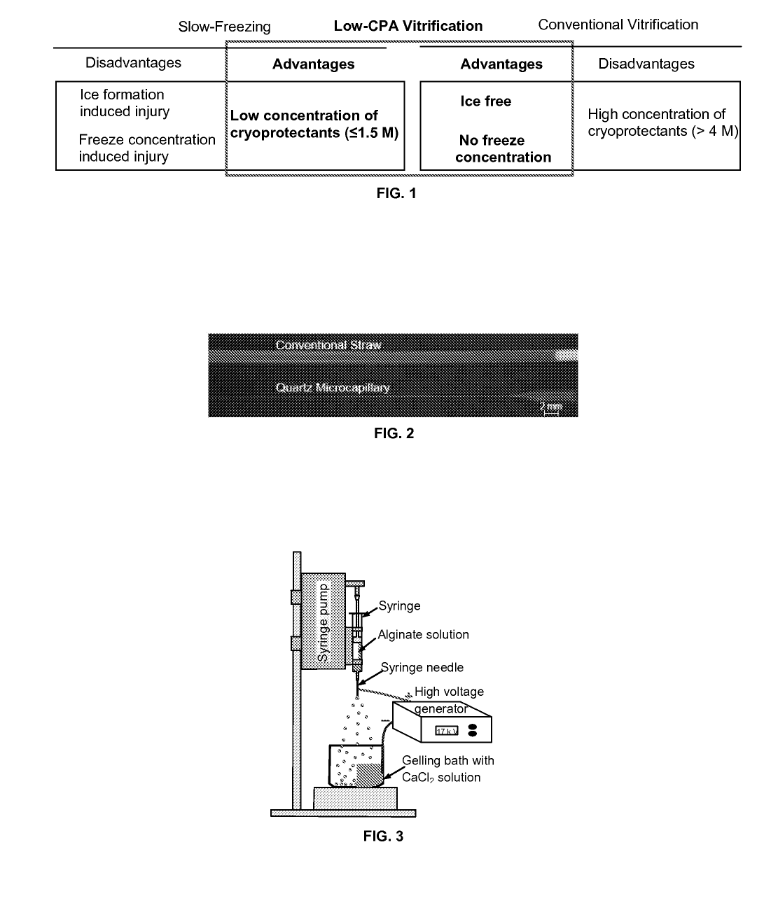

Low-CPA vitrification of mammalian cells with a significantly lower concentration (2 M) of intracellular cryoprotectants is achievable by using a thin quartz microcapillary. Theoretically, mammalian cells can be vitrified using a low, nontoxic concentration of cryoprotectants by ultrafast cooling (>100,000° C./min) the cells to a vitrified/glassy state at cryogenic temperatures (i.e., low-CPA vitrification that combines the advantages of the conventional slow-freezing and vitrification approaches while avoiding their shortcomings, FIG. 1). This is because the higher the cooling rate, the lower the cryoprotectant concentration that is required for vitrification. For example, even pure water can be vitrified when the cooling rate is higher than 106° C./s. In a recent study it has been demonstrated that an ultra-high cooling rate (˜200,000° C./min) can be achieved by plunging a quartz micro-capillary (inner diameter: 180 μm and wall thickness: 10 μm, FIG. 2) into liquid nitrogen. The present inventor has previously found that suspended single mouse ES cells can be successfully vitrified with this novel technique at an intracellular cryoprotectant (i.e., 1,2-propanediol or propylene glycol) concentration as low as 2 M, as further described in U.S. application Ser. No. 12/226,300, which is incorporated by reference herein. The present disclosure describes either further decreasing the required cryoprotectant concentration at the same cooling rates or decreasing the minimum cooling rates at the same cryoprotectant concentration for vitrifying the cells to a low, nontoxic level (i.e., ≦1.5 M) by combining the novel quartz microcapillary technology with cell microencapsulation.

Vitrification is a better approach than slow freezing particularly for cryopreservation of microencapsulated cells. Microencapsulated cells are notoriously difficult to cryopreserve by slow freezing because mechanical damage to microcapsules and the encapsulated cells as a result of ice formation is inevitable during the slow freezing process. For example, a dimethyl sulfoxide (DMSO) concentration of 2.8 M, which is much higher than that required for non-encapsulated single cells (i.e., ≦1.5 M, FIG. 1), has been shown to be necessary for cryopreservation of hepatocytes encapsulated in collagen hydrogel microcapsules of 357 μm by slow freezing. However, a significant amount (>40%) of microcapsules still lost integrity even under this optimal condition. On the other hand, damage to microcapsules has been shown to be negligible when cryopreserving the microencapsulated cells by the conventional vitrification approach using a combination of approximately 7 M ethylene glycol and 0.6 M sucrose. The superiority of the conventional vitrification to slow freezing for cryopreserving the microencapsulated hepatocytes presumably is due to the ice free nature of the vitrification approach. Nevertheless, further optimization of the vitrification protocol is necessary since a cryoprotectant concentration of 7 M can cause significant osmotic and metabolic injury to sensitive mammalian cells. Application of the low-CPA vitrification approach for cryopreserving microencapsulated living cells will reduce the necessary cryoprotectant concentration to a nontoxic level (≦1.5 M) as that used for slow freezing of single mammalian cells.

The present disclosure describes methods for the cryopreservation of mammalian cells that combines the advantages of the slow-freezing and conventional vitrification approaches while avoiding their shortcomings. For cryopreservation, the microencapsulated mammalian cells are placed in a vitrification solution that is then placed in the capillary tube. The capillary tubes used in the methods of the invention include a wall that is made of a thermally conductive material, where the ratio of the thermal conductivity of the wall to the wall thickness is at least 1,000, 5,000, 10,000, 100, 000, 500,000, or higher. The solution in the capillary tube is then exposed to temperatures equal to or less than −80° C., preferably equal to or less than liquid nitrogen or slush nitrogen temperature (e.g., −196° C. or −205° C., respectively) and the vitrification solution containing the microencapsulated mammalian cells is cooled at a rate greater than or equal to 30,000-100,000,000° C./minute, preferably 100,000° C./minute, 200,000° C./minute, 350,000° C./minute, 1,000,000° C./minute, or higher. The exposure of the highly conductive tube with the low thermal mass allows for vitrification of the solution in the absence of ice formation. Cyroprotectants can also be added to the vitrification solution to further prevent ice formation. The present methods are advantageous over the traditional methods for cryopreservation because they achieve rapid cooling in the absence of ice formation and toxic concentrations of cryoprotectants, both of which greatly increase the viability and usability of the cryopreserved cell after it has been warmed.

Cells

The present invention can be used for the cryopreservation of any type and any species of mammalian cells. For example, the method can be used to cryopreserve oocytes or sperm in assisted reproductive technology or for patients undergoing chemotherapy or radiation therapy. The method can also be used for the cryopreservation of stem cells, such as embryonic stem cells, or other cells, which can then be used as the basis of stem cell-based therapies, cell transplantation, tissue engineering, and regenerative medicine. The method can also be used to cryopreserve oocytes or sperm from an animal that is rare or at risk of becoming extinct for future use in assisted reproductive technologies for the preservation of the species. The method can further be used for animal husbandry purposes (e.g., the breeding and raising of animals), for example, for the cryopreservation of embryonic stem cells, gametocytes, oocytes, or sperm from animals such as cows, pigs, and sheep.

Cell types that may be cryopreserved using the compositions and methods of the present invention include, for example, differentiated cells, such as epithelial cells, cardiomyocytes, neural cells, epidermal cells, keratinocytes, hematopoietic cells, melanocytes, chondrocytes, B-cells, T-cells, erythrocytes, macrophages, monocytes, fibroblasts, or muscle cells; and undifferentiated cells, such as embryonic, mesenchymal, or adult stem cells. Additional cell types that can be cryopreserved using the methods of the invention include gametocytes, oocytes, sperm, zygotes, and embryos. The cells can be haploid (DNA content of n; where “n” is the number of chromosomes found in the normal haploid chromosomes set of a mammal of a particular genus or species), diploid (2 n), or tetraploid (4 n). Other cells include those from the bladder, brain, esophagus, fallopian tube, heart, intestines, gallbladder, kidney, liver, lung, ovaries, pancreas, prostate, spinal cord, spleen, stomach, testes, thymus, thyroid, trachea, ureter, urethra, or uterus.

The cells may be from a human or non-human mammal, for example Cercopithecoidea family, Hominoidea superfamily, Canis familiaris, Felis catus, Cricetidae spp., Equus spp (e.g., Equus caballus, Equus assinus), Equidae family, Bos taurus, Bos indicus, Bovidae family, Camelidae family, Bubalus bubalis, Capra aegagrus hircus, Cervidae family, Cervinae family, Ovis aries, Ovis canadensis, Capra hircus, Sus scrofa domestica, Mesocricetus spp., Mustela vison, Cavia porcellus, Meriones unguiculatus, Chinchilla laniger, Rattus norvegicus, Rattus spp., Mus musculus, Leporidae family, Oryctolagus cuniculus, Kobus spp., Gallus spp., Meleagria gallopavo, Anatidae spp., Mustela putorius, Columba domestica, Columba livia, Numida meleagris, Ornithorhynchus anatinus, Pavo cristatus, Bison spp., Struthio spp., Lama glama, Rhea spp., Dromiceius spp., Lama pacos, Rangifer tarandus, Bos grunniens, Camelus bactrianus, Camelus dromedarius), and any endangered or threatened species (e.g., those species identified by the U.S. Fish and Wildlife Service (USFWS) Threatened and Endangered Species System (TESS)).

The cells are prepared for cryopreservation by encapsulating the cells in microcapsules with the appropriate permeability to exclude immunocytes, antibodies, and complement factors that are generated by the host immune system while allowing controlled and sustained release of therapeutic products produced by the cells. In this regard, microcapsules made of biocompatible polymers are contemplated for use in connection with the present disclosure. For instance, natural biodegradable and biocompatible polysaccharides can be utilized with the present disclosure. One example of a suitable material is a microcapsule formed from alginate. Importantly, microcapsules suitable for use with the present disclosure are less than about 300 μm in diameter and, more particularly, can be less than 100 μm, such as less than 95 μm or less than about 90 μm in diameter. In certain embodiments of the present disclosure, microcapsules suitable for use with the present disclosure are less than about 75 μm in diameter and, more particularly, can be less than 50 μm or less than about 25 μm in diameter. Suitable mechanisms for forming such microcapsules include electrostatic spray methods or any other suitable mechanism as would be known in the art. Cells can be encapsulated in such microcapsules using methods as further described herein or any other suitable method as would be known in the art.

The encapsulated cells can be mixed with a vitrification solution. The encapsulated cells can be added to the vitrification solution or the vitrification solution can be added to the encapsulated cells. Alternatively, the cell media is modified to include all the components needed for vitrification. The concentration of the encapsulated cells in the vitrification solution varies widely depending on the cell type. For example, for oocytes, the concentration of cells can be low, for example, as low as 1 cell/ml, while for stem cells, the cell concentration can be higher than 10 million/ml. The exact concentration can be determined by the skilled artisan for the particular cell type.

The vitrification solution can include any physiologic solution such as 1× phosphate buffered saline (PBS), FHM (a flush hold Hepes-buffered medium from Specialty Media, Lavallette, N.J.), or a cell media, for example, a stem cell culture medium that includes Knockout DMEM+15% Knockout Serum Replacement (Invitrogen, Carlsbad, Calif.) containing 1000 U/ml Leukemia Inhibitory Factor (Chemicon, Temecula, Calif.). The vitrification solutions is desirably supplemented with one or more components including, but not limited to, serum, proteins, penicillin/streptomycin, lipids, salts, formamide, methoxylated compounds, polymers (e.g., polyvinyl pyrrolidone and polyvinyl alcohol), cryoprotectants, and/or sugars.

The encapsulated cells can then loaded into a capillary tube, for example by capillary action or using a syringe, and the tube is then exposed to temperatures less than or equal to −80° C., preferably less than or equal to the temperature of liquid nitrogen or slush nitrogen temperature (e.g., −196° C. or −205° C., respectively), and the vitrification solution containing the mammalian cells is cooled at a rate equal to or greater than 30,000-100,000,000° C./minute, for example, 100,000° C./minute, 200,000° C./minute, 350,000° C./minute, or even 1,000,000° C./minute. For example, the tube can be plunged into liquid nitrogen or slush or slurry nitrogen; optionally with shaking, to cause vitrification of the encapsulated cell and the vitrification solution in the absence of ice formation. The vitrified cells in the vitrification solution can then be stored at a temperature less than or equal to −80° C., preferably equal to or less than liquid nitrogen temperature for any desired amount of time (e.g., up to and beyond 10 years) until the cell is needed. The cells are then warmed, using any number of techniques known in the art, for example, by plunging the tube into a 1×PBS solution at 20-37° C., preferably room temperature, optionally with shaking and optionally supplemented with sugar or other CPAs. After warming, the cells are generally washed and treated as needed for the research or clinical applications. It will be clear to the skilled artisan the exact conditions and media that are used for culturing the cells before, during, and after warming. Exemplary protocols are provided in the examples below.

Capillary Tubes

The devices used in the methods of the present invention include capillary tubes with various dimensions and materials. Generally, the capillary tube is a microcapillary tube and is made of a wall material that is thermally conductive. Desirably, the microcapillary tube has thin walls (e.g., about 1 to 100 μm), where the ratio of thermal conductivity to wall thickness is at least 1,000, 5,000, 10,000, 100, 000, 500,000, or higher. Desirably, the microcapillary tube has a relatively small diameter (e.g., from 1 to 800 μm). In one example, the microcapillary tube is a quartz microcapillary tube (QMC), commercially available, for example from Charles Supper Co. (Natick, Mass.) or Wolfgang Muller Glas Technik (Germany). The combination of a highly conductive material with thin walls and a relatively small diameter of the tube allows for rapid cooling in the absence of ice formation or toxic levels of CPAs.

Material

The vitrification devices of the present invention can be made of any thermally conductive material. Preferably, the material has a thermal conductivity greater than 5 W m⁻¹ K⁻¹, preferably greater than or equal to 8 W m⁻¹ K⁻¹. Examples of thermally conductive materials useful for the capillary tubes in the methods of the invention include plastics, glass, quartz, sapphire, gold, copper, silver, diamond, titanium, palladium, platinum, and stainless steel.

Cryoprotective Agents (CPAs)

As described above, the present methods feature the cryopreservation of mammalian cells by vitrification in the absence of ice formation or toxic levels of cryoprotectants. The vitrification solution can include CPAs, which are preferably included at concentrations less than about 1.5 M, less than 1.4 M, 1.3 M, 1.2 M, 1.1 M, 1.0 M, or 0.5 M. The CPAs can be permeating or non-permeating or a combination of permeating and non-permeating. Exemplary threshold concentrations of CPAs as a percentage of volume are described in detail in the examples below.

Non-limiting examples of CPAs that are useful in the methods of the invention include sugar, polypropylene glycol, ethylene glycol, 1,2-propanediol (PROH), glycerol, and DMSO. Desirably the sugar is any one of the following: sucrose, trehalose, raffinose, stachyose, and dextran. Exemplary sugars and the concentration ranges for such sugars are described in U.S. Pat. Nos. 6,673,607 and 7,094,601, herein incorporated by reference.

CPAs can be added to the vitrification solution as a single agent or as a combination of one or more agents. For example, 1.5M ethylene glycol or 1,2-propanediol (PROH) can be supplemented with 0.5 to 2M sugar to produce a synergistic effect. For example, a combination of ethylene glycol and a sugar or a combination of PROH and a sugar can be used. In one example, 1.5M PROH and 0.5 M trehalose are added to the vitrification solution. In another example, 0.3M sucrose and 1.5M PROH is used. The combination of a permeating and non-permeating CPA allows for a lower intracellular concentration of CPA, since the non-permeating CPA does not enter the cell. For example, in the 1.5M PROH and 0.5 M trehalose example described above, the intracellular concentration of CPA would be 1.5M since trehalose is not permeable to the plasma membrane of mammalian cells.

Nanoparticles and Microparticles

The vitrification solution can further include nanoparticles or microparticles or both. The addition of nanoparticles, microparticles, or nanotubes are is thought to enhance the thermal conductivity of the vitrification solution. Examples of such nanoparticles or micro particles include particles having carbon or a noble metal, such as gold, silver, titanium, palladium, platinum, or similar particles thereto. Examples of such nanoparticles and/or microparticles and/or nanotubes may include, but are not limited to, carbon or noble metals, e.g., gold, silver, titanium, palladium, platinum, and copper.

In one aspect of the invention, the nanoparticles are present in the vitrification solution in an amount up to 99%, 50%, 25%, 20%, 10%, 5% or lower, based on the total weight of the solution. In another aspect of the present disclosure, the microparticles are present in the vitrification solution in an amount up to 99%, 99%, 50%, 25%, 20%, 10%, 5%, based on the total weight of the solution. It has been shown that the presence of a small fraction (<1% vol) of nanoparticles in a solution can increase the thermal conductivity of the solution up to more than 200% (Choi et al., Applied Physics Letter 79: 2252-2254, 2001; Eastman et al., Applied Physics Letter 78: 718-720, 2001).

Surface Techniques

The outer surface of the capillary tubes can be treated to enhance the boiling heat transfer coefficient on the boundary between the cryogenic fluid/slush/slurry (i.e., liquid nitrogen or its slush) and capillary. Such techniques include microfins and surface coating. Microfins can increase the surface to volume ratio to enhance heat transfer. The use of such coatings can, for example, reduce the bubble formation or bubble attachment on the surface and hence enhance heat transfer between the cryogenic fluid/slush/slurry and capillary tube.

Ice Blockers

Ice blockers can also be included in the vitrification solution. Non-limiting examples of ice blockers include polymers and peptides having properties that inhibit ice nucleation and growth within the medium or similar polymers and peptides thereto. Examples of such polymers and peptides may include, but are not limited to, polyvinyl alcohol, polyglycerol, antifreeze proteins, and other polymer and peptides referred to in the art as ice blockers.

Cooling the Cells

The methods of the invention include exposing the capillary tube housing the vitrification solution and the mammalian cells to a temperature equal to or less than −80° C. (dry ice temperature), preferably less than or equal to liquid nitrogen temperature or −196° C. or slush nitrogen (˜205° C.), which is a mixture of liquid and solid nitrogen. Generally, the capillary tube will be plunged into the cryogenic fluid/slush/slurry, for example, the liquid or slush nitrogen. Optionally, the method can include shaking the capillary tube at a frequency of about 1.0 Hz or higher while it is being exposed to the cryogenic temperatures (e.g., less than or equal to −80° C.).

Viability of Cryopreserved Cells

When desired, the cryopreserved mammalian cells of the invention can be warmed, using methods known in the art or described herein. For example, the capillary tube can be plunged into a 1×PBS solution at 20-37° C., preferably room temperature, optionally with shaking and optionally supplemented with sugar or other CPAs. After warming, the cells are generally washed, suspended in the appropriate media and treated as needed for use in research or clinical applications. For example, embryonic stem cells can be plated and passaged using techniques known in the art. Oocytes are generally cultured in droplets of media immersed in oil. It will be clear to the skilled artisan the exact conditions and media that are used for culturing the cells before and after cryopreservation.

There are various tests known in the art to determine the viability and function of the cells after warming and these tests are dependent on the cell type and the desired use of the cell. For example, for ES cells that are to be used for cell-based therapeutics, maintenance of pluripotency is very important. The pluripotency of the ES cells can be tested using art known methods, including, for example, Oct4-GFP expression, elevated alkaline phosphatase expression, and SSEA-1 surface glycoprotein expression. The ability of cells to attach efficiently is another assay for the viability and usability of many cells. Attachment assays are known in the art and described herein. Proliferation assays can also be used to determine if the attached cells can proliferate as expected after cryopreservation. Attachment and proliferation efficiency can be compared to control cells, which have not undergone cryopreservation. For cryopreservation of zygotes, cleavage rates can be determined after cryopreservation and compared to control groups to determine if there has been any cellular damage during the cryopreservation process. The viability of oocytes can be determined by examination of the morphological characteristics of the cells following cryopreservation. Morphologically viable oocytes exhibits intact zona pellucida and plasma membrane and refractive cytoplasm, while non-viable oocytes appear degenerated when visualized under a light microscope. The ultimate criterion for oocyte viability and function is their capability to be fertilized by healthy sperm in vitro and in vivo, followed by cleavage, blastocyst, and/or hatching or development of the fetus.

The assays for cell viability, function, and usability can also be used to test parameters for the cryopreservation methods described herein. For example, variations in CPAs or CPA concentrations or in cooling rates or capillary tube materials or dimensions can readily be tested on cells and their effects on cell viability, function, and usability can be tested using any of the methods described herein or known in the art.

Reference now will be made to exemplary embodiments of the invention set forth below. Each example is provided by way of explanation of the invention, not as a limitation of the invention.

### Example 1

The present example achieves effective microencapsulation of mouse embryonic stem (ES) cells in small microcapsules (˜100 μm) and cryopreservation of the microencapsulated cells by low-CPA vitrification with an intracellular cryoprotectant concentration up to 1.5 M. To date, studies have: 1) successfully determined the optimal conditions for microencapsulating mouse mesenchymal stem cells in small alginate microcapsules (˜100 μm) and 2) successfully cryopreserved non-microencapsulated mouse ES cells by low-CPA vitrification using an intracellular cryoprotectant concentration as low as 2 M. A more detailed description of the methods used and results obtained in these studies is given below.

Effects of Preparation Parameters on the Size and Morphology of Small Alginate Microcapsules Prepared Using the Electrostatic Spray Method

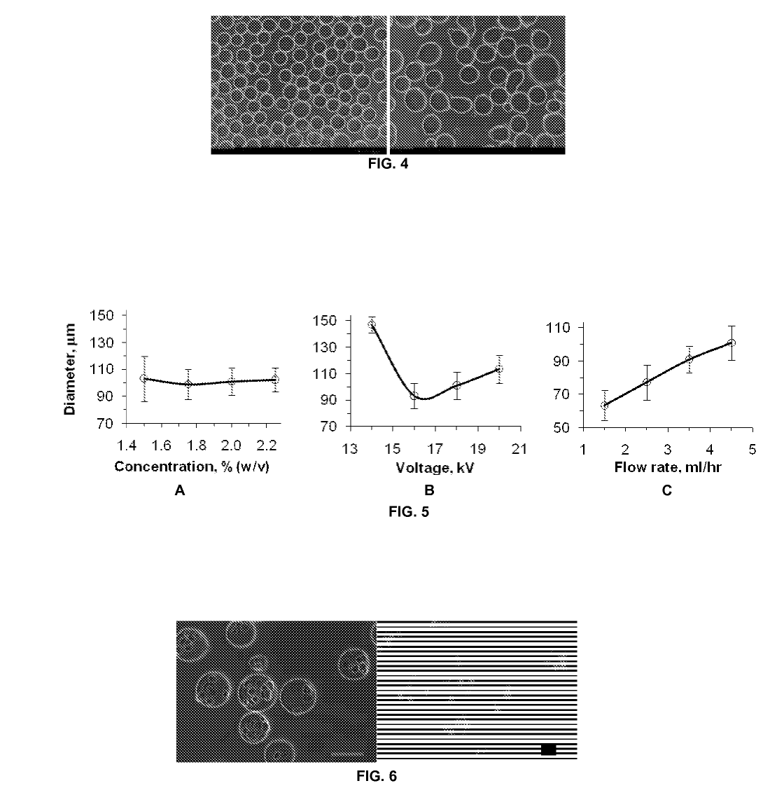

Small (˜100 μm) cell-loaded alginate microcapsules using the electrostatic spray method have been successfully prepared. Purified alginate (type M) (Medipol, Lausanne, Switzerland) was used because of its excellent biocompatibility. The type M alginate with a low guluronic acid content was selected because it has been demonstrated to result in smaller microcapsules than alginate with a high concentration of the acid under similar preparation conditions. A schematic of the electrostatic spray device is given in FIG. 3. Alginate microcapsules were generated by extruding sodium alginate solution into 1.2 (w/v) of calcium chloride solution through a blunt syringe needle driven by a syringe pump. The inner diameters and wall thickness of the syringe needle used are 150 and 75 μm, respectively. The distance between the needle tip and the free surface of the calcium chloride solution in the gelling bath was set at 2.5 cm. During extrusion, droplets are formed on the tip of the blunt needle and sprayed into the calcium chloride solution (gelling bath) as a result of multiple forces including the driven force of syringe pump, electrostatic force (due to the high electrostatic potential applied between the gelling bath and the needle), surface tension, and gravity. We first investigated the effect of the three major preparation parameters (i.e., the sodium alginate concentration, spray voltage, and flow rate) on microcapsule size and morphology in the absence of living cells. Typical images of the microcapsule with good (round in shape and uniform in size) and bad (irregular in shape and non-uniform in size) morphology are shown in panels A and B of FIG. 4, respectively. Typical data showing the effect of the preparation parameters on microcapsule size is given in FIG. 5. FIG. 5A shows that the microcapsule size does not change significantly when the sodium alginate concentration varies from 1.5 to 2.25% (w/v). However, the microcapsules become irregular in shape and non-uniform in diameter when the sodium alginate concentration is not higher than 1.5% (w/v). FIG. 5B shows that the spray voltage greatly affects the microcapsule size in a non-linear manner: the microcapsule diameter decreases first, reaches a minimum, and then increases slightly with the increase of spray voltage from 14 to 20 kV. The optimum voltage for producing small microcapsules with good morphology is from 16-18 kV. FIG. 5C shows that the microcapsule size increases monotonically with the increase of spray flow rate from 1.5 to 4.5 ml/hr. Although the spray flow can become discontinuous when the flow rate is lower than 1.5 ml/hr, microcapsules with good morphology could be generated when it is between 1.5 and 4.5 ml/hr. In summary, to obtain small microcapsules (˜100 μm) with good morphology, the alginate concentration should be higher than 1.5% (w/v) and the spray voltage and flow rate should be at least 16 kV and 1.5 ml/hr, respectively.

Encapsulation of Mouse Mesenchymal Stem Cells in Small (˜100 μm) Alginate Microcapsules C3H10T1/2 mouse mesenchymal stem cells (ATCC, Manassas, Va.) have been successfully encapsulated in alginate microcapsules of ˜100 μm using the electrostatic spray method. C3H10T1/2 cells were cultured in DMEM (Invitrogen, Carlsbad, Calif.) with 10% fetal bovine serum, 100 U/ml penicillin and 100 mg/l streptomycin (Hyclone, Logan, Utah) in a humidified 5% CO2 incubator at 37° C. For cell microencapsulation studies, C3H10T1/2 cells were detached from culture dishes with trypsin/EDTA digestion (Invitrogen, Carlsbad, Calif.), centrifuged for 5 minutes at 1200 rpm, and resuspended in 0.9% (w/v) sodium chloride solution. Cells were then centrifuged and resuspended in sodium alginate solution. The cell suspension was transferred to a 1 ml syringe for producing cell-loaded alginate microcapsules using the electrostatic spray device (FIG. 3). The goal for the cell microencapsulation study is to determine the optimal preparation parameters of the electrostatic spray method for producing small (˜100 μm) cell loaded microcapsules with not only good morphology but also high cell encapsulation efficiency (i.e., number of cells per microcapsule). Preliminary studies show that microcapsules prepared in the presence of living cells are always bigger than that produced in the absence of cells under the similar preparation conditions. Therefore, the three preparation parameters discussed in the previous section need to be further optimized for generating small (i.e., ˜100 μm) cell encapsulated microcapsules. Since the alginate concentration does not affect the microcapsule size significantly according to FIG. 5A, a 2% (w/v) alginate solution was used for all cell encapsulation studies to ensure good cell morphology. An additional preparation parameter for cell encapsulation is cell density in sodium alginate solution. Preliminary studies show that the microcapsule morphology could be significantly compromised (i.e., become irregular and non-uniform) when the cell density is more than 5×106 cells/ml. On the other hand, many microcapsules without cells were generated if cell density is less than 3×106 cells/ml.

Based on these observations and the data shown in FIG. 5, a total of nine experimental conditions were determined to be necessary for optimizing the three parameters for preparing small cell-loaded microcapsules using the orthogonal array testing method. Table 1 shows the 9 experimental conditions together with the experimental results of microcapsule size prepared in the presence of living cells and cell encapsulation efficiency (i.e., cells per microcapsule). The data in Table 1 were further analyzed using the F test and an F value greater than 9 (or p<0.1) was taken to be statistically significant. In addition to spray flow rate (F=14.741), cell density was identified to affect the microcapsule size significantly (F=12.259). However, cell density was identified to be the dominant factor that significantly affects the encapsulation efficiency (F=15.221) among the three preparation parameters over the ranges given in Table 1. In summary, to prepare small cell-loaded microcapsules of ˜100 μm with good

TABLE 1
Experimental design (i.e., 9 conditions) determined using the orthogonal array
testing method and the corresponding experimental results (mean ± SD) [18]

| Experimental Conditions | | | | Experimental Results | |
|---|---|---|---|---|---|
| | Cell Density | Spray | Flow Rate | Microcapsule Size | Encapsulation Efficiency |
| Condition | (10⁶ cells/ml) | Voltage (kV) | (ml/hr) | (μm) | (cells/microcapsule) |
| 1 | 1 | 16 | 1.5 | 70.94 ± 13.87 | 0.95 ± 1.09 |
| 2 | 1 | 18 | 2.0 | 81.69 ± 12.79 | 1.75 ± 1.54 |
| 3 | 1 | 20 | 3.0 | 104.45 ± 12.79 | 1.42 ± 1.35 |
| 4 | 3 | 16 | 3.0 | 83.16 ± 19.35 | 4.92 ± 1.84 |
| 5 | 3 | 18 | 1.5 | 114.46 ± 10.62 | 3.95 ± 1.83 |
| 6 | 3 | 20 | 2.0 | 97.10 ± 10.96 | 2.78 ± 1.58 |
| 7 | 5 | 16 | 2.0 | 127.02 ± 11.33 | 10.13 ± 2.65 |
| 8 | 5 | 18 | 3.0 | 96.01 ± 18.12 | 5.32 ± 2.20 |
| 9 | 5 | 20 | 1.5 | 113.70 ± 16.89 | 5.55 ± 2.19 |

morphology, the sodium alginate concentration should be higher than 1.5% (w/v), the spray voltage should be 16-18 kV, the spray flow rate should be 1.5-3 ml/hr, and the cell density in the sodium alginate solution should be 3-5×106 cells/ml.

A typical image of the small cell loaded microcapsules prepared under an optimal condition (2% (w/v) of sodium alginate solution, 17 kV of spray voltage, 2 ml/hr of spray flow rate, and 4.5×106 cells/ml of cell density) is shown in FIG. 6A. The microcapsule diameter was determined to be 97.2±11.52 μm with an encapsulation efficiency of 8.08±2.82 cells per microcapsule. The immediate viability of the encapsulated cells was studied using the standard live/dead assay from Invitrogen (Carlsbad, Calif.) to stain viable cells as green and dead cells as red (FIG. 6B). The cell viability after microencapsulation was quantified to be 89.2±1.2% indicating that cells can survive the microencapsulation procedure by electrostatic spraying well.

Cryopreservation of Mouse Es Cells by Ultrafast Cooling Using Quartz Microcapillaries

A novel ultrafast cooling (cooling rate >2×10⁵° C./min) technique for ice-free cryopreservation (i.e., ultrafast vitrification) of mouse ES cells has been recently developed using quartz microcapillaries (180 μm in inner diameter and 10 μm in wall thickness in the working part). A comparison of the quartz microcapillary and the traditional French type plastic straw used for cell vitrification is given in FIG. 2. With this quartz microcapillary technique, the required concentration of cell membrane permeable cryoprotectants (i.e., 1,2-propanediol or PROH) can be reduced from 4-6 M to 2 M. It was successfully applied for cryopreserving R1 mouse ES cells (ATCC, Manassas, Va.) which express green fluorescent protein (GFP) under the control of an Oct-4 promoter. The ES cells were cultured in a maintenance medium consisted of Knockout DMEM supplemented with 15% knockout serum replacement (Invitrogen, Carlsbad, Calif.) containing 1000 U/ml LIF (Chemicon, Temecula, Calif.). Feeder layer-free ES cells were continually passaged in 0.1% gelatin-coated 75 cm2 T-flasks in 5% CO2 humidified air at 37° C. For cryopreservation experiments, the cells were loaded with cryoprotectants in two steps: 1) incubating in a solution made of ES cell maintenance medium with 1.5 M 1,2-propanediol for 10 min and 2) incubating in a solution made of ES cell maintenance medium with 2 M 1,2-propanediol and 0.5 M trehalose for another 10 min. The cell suspension was then loaded into quartz microcapillaries and cooled by plunging the microcapillaries into liquid nitrogen. The cryopreserved cell suspension was warmed by plunging the microcapillaries into 1×PBS with 0.2 M trehalose at room temperature. The cell suspension was then unloaded from the microcapillaries and further processed for the assessment of immediate (i.e., within 3 hr at day 0, the day of experiments) viability, attachment efficiency (representing viability at day 1 post cryopreservation), proliferation/growth, and analysis of the mouse ES cell specific indicators of pluripotency. Fresh cells and cells cryopreserved with either 0.5 M extracellular trehalose or 2 M 1,2-propanediol alone were studied as controls.

Viability of Mouse Embryonic Stem Cells Post Cryopreservation by Ultrafast Cooling

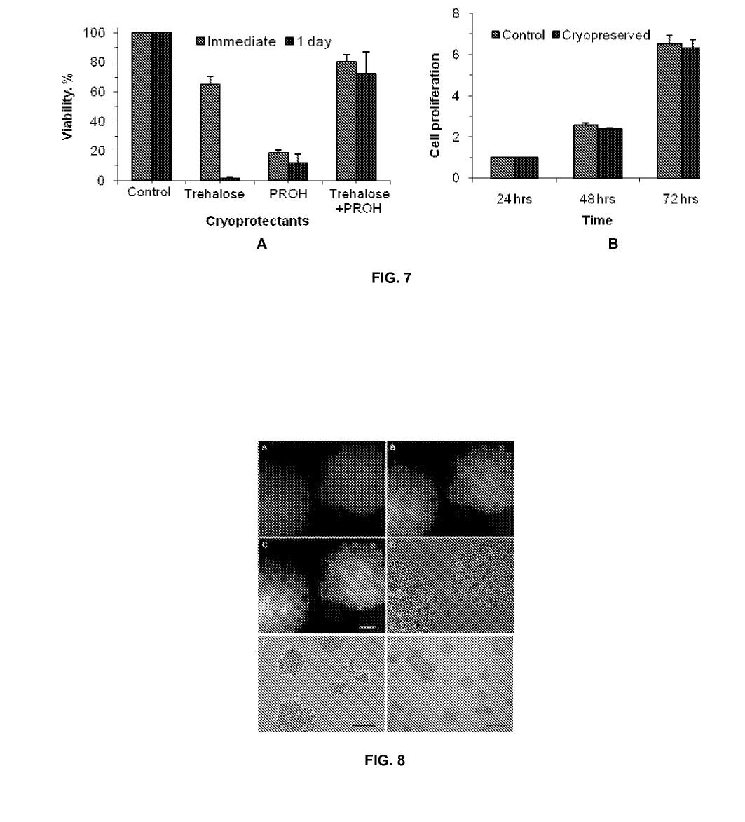

The immediate cell viability post cryopreservation represented by membrane integrity was assessed using the standard live/dead assay kit of two fluorescent probes: calcein AM and ethidium homodimer (Invitrogen, Carlsbad, Calif.). Cells that excluded ethidium homodimer and retained the fluorescent calcein were counted as being viable. To check cell viability more than one day post cryopreservation, cell attachment evaluated at day 1 and proliferation/growth evaluated over a three-day observation period were also studied. To do this, the cells after cryopreservation were unloaded with cryoprotectants, resuspended in 1.5 ml fresh ES cell maintenance medium, and cultured in a 33 mm Petri dish coated with 10 μg/ml fibronectin (Chemicon, Temecula, Calif.). Fresh cells without cryopreservation were seeded at the same cell concentration as control. The number of cells in at least 20 randomly selected representative fields under a 10× objective was counted every day for all the samples. Proliferation was assessed by normalizing the number of cells at day 3 and day 2 to that at day 1. Attachment efficiency (i.e., viability at day 1) was calculated as the ratio of the number of cells of a cryopreserved sample to that of the control non-frozen sample at day 1. The immediate and 1 day cell viability post cryopreservation using various cryoprotectants are shown in FIG. 7A. Only a small percentage of cells (˜20%) can survive the cryopreservation procedure using 2 M 1,2-propanediol (PROH) as the sole cryoprotectant. When adding 0.5 M trehalose into the vitrification solution, however, the immediate cell viability increased to more than 80%, even though trehalose could not permeate the cell plasma membrane and was present only extracellularly. The immediate cell viability for cryopreservation using 0.5 M extracellular trehalose as the sole cryoprotectant is about 65%. Unlike the immediate viability, only a minimal number of cells were able to survive at day 1 (i.e., attach to substrate) when using 0.5 M extracellular trehalose (<2%) as the sole cryoprotectant during vitrification (FIG. 7A). This result indicates the necessity of intracellular cryoprotectant to provide protection from within the cells during cryopreservation. Similarly, only a minimal number of cells were able to attach when using 2 M PROH (˜12%) as the sole cryoprotectant (FIG. 7A, 1 day viability). The 1 day viability, however, was much higher (˜72%) when the cells were cryopreserved using the combination of 0.5 M extracellular trehalose and 2 M cell membrane permeable 1,2-propanediol. Therefore, 1,2-propanediol and extracellular trehalose appear to have a synergistic effect on protecting ES cells from damage during cryopreservation. The proliferation/growth of the attached ES cells post cryopreservation using the combination of 0.5 M trehalose and 2 M PROH was very similar to that of the control non-frozen cells over a three day period (FIG. 7B). Therefore, the ice free ultrafast vitrification cryopreservation procedure did not appear to affect the growth characteristics of the mouse ES cells.

Undifferentiated Properties of Mouse Es Cells Post Ultrafast Vitrification

To determine whether the mouse ES cells retained their undifferentiated properties post vitrification, three different indicators that are characteristic to the cells were studied: expression of transcription factor Oct-4, expression of membrane surface glycoprotein SSEA-1, and the elevated expression of alkaline phosphatase. For immunofluorescence staining of SSEA-1, ES cells were fixed using 4% paraformaldehyde, permeabilized with 0.4% Triton X-100, and blocked against non-specific binding with 2% BSA and donkey serum. Monoclonal antibody against SSEA-1 (clone MC-480) was purchased from Chemicon (Temecula, Calif.). Antibody localization of SSEA-1 was performed using a Texas Red conjugated goat anti-mouse F(ab′)₂ fragment antibody (Rockland, Gilbertsville, Pa.). Mounting medium containing DAPI (Vector Labs, Burlingame, Calif.) was also applied to the cells before observation. Histochemical staining of alkaline phosphatase was performed by incubating naphthol AS-BI phosphate and fast red violet solutions (Chemicon, Temecula, Calif.) with 4% paraformaldehyde-fixed ES cells for 15 min. Undifferentiated properties of the ES cells was verified by high levels of staining for the membrane surface glycoprotein SSEA-1 (FIG. 8A) and expression of the green fluorescent transcription factor OCT-4 (FIG. 8B). The merged view of the red (SSEA-1), green (OCT-4), blue (DAPI) channels indicates extensive co-expression of the two markers overlapping with the cell nuclei (FIG. 8C). Phase contrast image (FIG. 8D) shows cells with high nuclei/cytoplasm ratios and compact colony formation typical of pluripotent ES cells. Histochemical staining shows strong expression for alkaline phosphatase at high magnification (FIG. 8E) which was seen to be well distributed within each colony as observed at a lower magnification (FIG. 8F). The positive expression and staining of the different markers that are characteristic to mouse ES cells suggests that the ES cells retained their undifferentiated properties post cryopreservation by ultrafast vitrification.

Microencapsulation of Mouse Embryonic Stem Cells in Small (˜100 μm) Alginate Microcapsules

Embryonic stem (ES) cells, pluripotent cells that can differentiate into a variety of somatic cell types, are becoming increasingly important for modern cell-based medicine due to the limited availability of non-proliferating cells. The full differentiation potential of ES cells, however, is induced by not only an appropriate biochemical environment but also the formation of multicellular aggregates of ES cells or embryoid bodies (EB). Traditional methods for EB formation (i.e., the hanging drop or spontaneous aggregation of ES cells in suspension approaches) are limited in their production capacity and difficulty to control the size and differentiation state of the produced EBs. Several recent studies have shown that microencapsulation of ES cells in alginate-based microcapsules results in the formation of ES cell aggregates of uniform size and provides a scalable system for mass production of EBs. Moreover, microencapsulation allows a better control of the biochemical microenvironment (or niche) for EB formation and the subsequent differentiation of non-autologous ES cells in vivo by immunoisolation of the encapsulated cells. However, the microcapsules used for encapsulating ES cells are more than 300 μm in all the reported studies. Considering that clinical practice of microencapsulation technology has been limited by inadequate transport of nutrients and metabolites to and from the microencapsulated cells and transplantation sites of large capsules, it is desired to use microcapsules as small as possible. Therefore, we propose to encapsulate mouse ES cells for EB formation and the subsequent differentiation in much smaller microcapsules (˜100 μm), which has been successfully prepared using the electrostatic spray method to encapsulate mouse mesenchymal stem cells as described above.

R1 mouse ES cells can be cultured in the same way as that described previously herein. The cells can be encapsulated in alginate microcapsules of approximately 100 μm using the electrostatic spray method. The optimized preparation parameters of the electrostatic spray method for producing small mesenchymal stem cell-loaded microcapsules shown in FIG. 6 can be used for generating the ES cell-loaded microcapsules, viz, spray voltage: 17 kV, spray flow rate: 2 ml/hr, cell density: 4.5×10⁶ cells/ml, and alginate concentration: 2% (w/v). The microencapsulated ES cells can be analyzed in terms of immediate viability, proliferation/EB formation, and preservation of pluripotent properties.

To study the immediate survival of ES cells post microencapsulation, the cells can be released from the microcapsules by chelating calcium cations in 0.055 M sodium citrate solution for 1 minute and collected by centrifuging. The cells will then be incubated at 37° C. for 15 minutes in culture medium with 9 μM calcein and ethidium homodimer included in the standard live/dead assay kit from Invitrogen (Carlsbad, Calif.), respectively. Calcein is hydrolyzed to form fluorescent products that can be retained only in live cells while ethidium homodimer can only enter and bind to the nucleus of cells with compromised membrane (taken as dead).

For cell proliferation study, the microencapsulated cells can be further divided into two groups and processed differently for further culture up to three weeks. The first group can be put in culture without any further processing. Microcapsules in the second group can be liquefied by calcium ion chelation in 0.055 M sodium citrate solution for 1 minute and the cells collected for further culture. In addition, the proliferation of fresh ES cells can be studied in parallel as control. Cell proliferation can be measured using the Alarmar blue assay (Biosource International, Camarillo, Calif.), which can be added to cell culture medium to monitor the dynamic proliferation and metabolic activity of microencapsulated cells in culture over a long period of time. The dynamics of EB formation can be monitored by quantifying the total number of EBs formed and their size and size distribution using confocal microscopy every day for three weeks.

Assessment of Pluripotency Pluripotent properties of the microencapsulated ES cells with good viability and normal proliferation (i.e., formation of EBs) at around two weeks can be studied in terms of expression of the mouse ES cell specific indicators including Oct-4, SSEA-1, and the elevated expression of alkaline phosphate as described herein. The Pluripotency of the ES cells can be further evaluated by their capability of osteogenic differentiation, which can be induced by culturing the EBs in the ES cell maintenance medium containing 1% antimycotic, 100 nM dexamethasone, 10 mM b-glycerolphosphate, and 0.05 mM L-ascorbic acid 2-phosphate. Alkaline phosphatase (ALP) activity, which is indicative of osteoblastic differentiation, can be assessed by histochemical staining as described herein and further quantified spectrophotometrically. The deposition of a hydroxyapatite matrix can be detected using the standard von Kossa stain Kit (DBS, Pleasanton, Calif.).

In accordance with the present example, ES cells can be encapsulated in alginate microcapsules of ˜100 μm or less with high immediate viability using the electrostatic spray method since the ES and mesenchymal stem cells are not very different in size (10-15 μm in suspension). Due to the small size to facilitate transport of nutrient, oxygen and metabolites, it is also expected that the ES cells can proliferate normally, form EBs of uniform size, and will retain their pluripotency when cultured in the small microcapsules. It has been reported that proliferation of mouse ES cells in the alginate matrix of microcapsules greater than 300 μm can be significantly slowed down when the sodium alginate concentration is more than 1.6% (w/v), which might be due to inadequate transport of oxygen, nutrients and metabolites and could be overcome by using the smaller microcapsules (˜100 μm). Otherwise, the small ES cell-loaded microcapsules can be prepared using a lower sodium alginate concentration (e.g., 1.75%, 1.55%). A high sodium alginate concentration is preferred because the harder microcapsules prepared using a higher concentration of sodium alginate are easier to cryopreserve by vitrification as a result of its high viscosity as can be discussed in more detail herein. Moreover, the surface of the ES cell-loaded microcapsules prepared with a high sodium alginate concentration (i.e., 2 and 1.75%) can be chemically cross-linked with poly-1-lysine (PLL, Mw=29 kDa, Sigma) by incubating the microcapsules in 0.05% (w/v) PLL solution for 5 minutes to form a solid-like semi-permeable membrane, followed by calcium chelation in 0.055 M sodium citrate solution to liquefy the alginate core. The liquid core of the resultant core-shelled microcapsules has been shown to facilitate ES cell proliferation/EB formation.

Low-CPA vitrification of mouse embryonic stem cells in small (˜100 μm) Alginate Microcapsules Using a Cryoprotectant Concentration No Higher than 1.5 M

Cell injury during cryopreservation can generally be attributed to two mechanisms: intracellular ice formation (IIF) and cell dehydration due to freeze-concentration of extracellular solution. During ultrafast cooling, however, the effect of cell dehydration is minimal since the cooling is so fast that intracellular water has no time to leak out before it is vitrified at a cryogenic temperature. Consequently, the dominant mechanism of cell injury during ultrafast cooling is intracellular ice formation, which could be minimized by cell microencapsulation for at least two reasons. First, the solid like alginate hydrogel in the microcapsule has a much higher viscosity than liquid solution, which should facilitate vitrification by preventing not only ice nucleation and growth in the microcapsule but also ice propagation from the bulk freezing solution into the microcapsules and the encapsulated cells. Second, since the alginate microcapsule can prevent ice propagation through its space, the encapsulated ES cells are essentially separated from the bulk solution in terms of the probability of ice formation. Consequently, the isolated sub-femtoliter (˜0.5×10⁻¹⁵ liters assuming a 10 μm diameter) cytoplasm of the encapsulated ES cells should be much easier to vitrify than that of a suspended ES cell having direct contact with the bulk solution. This is because it is generally much easier to vitrify solutions in a much smaller volume. The ability of alginate hydrogel microcapsules to facilitate vitrification is significant because it is expected that the required intracellular cryoprotectant concentration for vitrifying microencapsulated ES cells can be further reduced to a low, nontoxic level (i.e., 1.5 M) when used together with the quartz micro-capillary based ultrafast vitrification technique recently developed.

Mouse ES cells encapsulated in small alginate microcapsules prepared using the highest possible sodium alginate concentration for normal cell proliferation and preservation of pluripotency determined herein, will be further vitrified using quartz microcapillaries (180 μm in inner diameter and 10 μm in wall thickness). The small molecular weight 1,2-propanediol will be used as the cell membrane permeable cryoprotectants since it has been shown to be superior to other permeable cryoprotectants such as ethylene glycol and dimethylsulfoxide in terms of the vitrification capability. The concentration of 1,2-propanediol to be tested will be 1, 1.25, 1.5, 1.75, and 2 M. The disaccharide trehalose has been shown to facilitate vitrification when used together with 1,2-propanediol even though it is present only extracellularly. A trehalose concentration of 0.2 M will be used in the bulk vitrification solution. Mouse ES cells post vitrification can be evaluated in terms of their immediate viability, long-term proliferation/EB formation, and preservation of pluripotent properties in the same way as that described herein to assess the efficacy of the vitrification procedure.

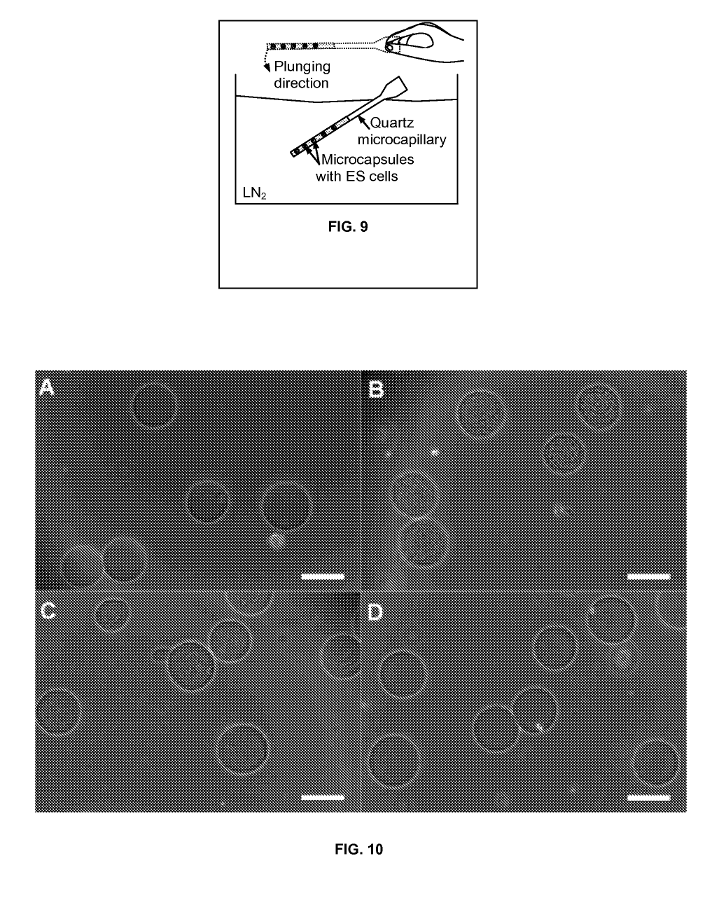

For vitrification studies using 1.75 or 2 M 1,2-propanediol, microencapsulated ES cells can be first incubated in maintenance medium with 1.5 M 1,2-propanediol for 15 min, followed by transferring to the medium with 1.75 or 2 M 1,2-propanediol and 0.2 M trehalose for another 15 min before loading into quartz micro-capillaries for vitrification. Otherwise, the microencapsulated cells can be incubated in the medium with the desired concentration of 1,2-propanediol (i.e., 1, 1.25, or 1.5 M) and 0.2 M trehalose for 15 min followed by loading into quartz microcapillaries for vitrification. The microencapsulated cells can also be cryopreserved using either 0.2 M trehalose or the desired amount of 1,2-propanediol (i.e., 1, 1.25, 1.5, 1.75, and 2 M) alone to serve as controls. The solution with microencapsulated ES cells in the quartz microcapillary can be cooled by plunging the microcapillary into liquid nitrogen using a technique that has been found to be the most effective for vitrification (FIG. 9): the quartz microcapillary is held above the liquid nitrogen (LN2) with its axial axis parallel to the free LN2 surface and the working part of the microcapillary loaded with microencapsulated cells is plunged into liquid nitrogen by rotating the microcapillary downward around the operator's wrist with a speed at the end of the thin tip greater than 1 m/s. The microcapillary was then left in liquid nitrogen for at least 3 hours, which is long enough because cell injury associated with cryopreservation occurs dominantly during the cooling and warming steps rather than during storage in LN2 when all chemical processes are essentially arrested as a result of the extremely low temperature. After storage, the cryopreserved ES cells can be warmed by plunging the quartz micro-capillary into 1×PBS with 0.2 M trehalose at room temperature in a manner similar to cooling as shown in FIG. 9. The cell loaded microcapsules will then be unloaded from the quartz microcapillary by pushing warm (37° C.) maintenance medium with 0.2 M trehalose from the large end of the quartz microcapillary using a 1 ml syringe with a soft polymer needle. The yield of this unloading procedure was found to be more than 90% when the suspension of non-microencapsulated ES cells in the microcapillary was collected into a 10 μl droplet after vitrification. The microencapsulated ES cells unloaded from the microcapillary will then be incubated in maintenance medium with 0.2 M trehalose for 10 minutes, centrifuged, and transferred into fresh culture medium for 10 minutes to remove extracellular trehalose. The cells can then be processed for further analysis of immediate viability, long-term proliferation/EB formation, and preservation of pluripotent properties as described herein for microencapsulating mouse embryonic stem (ES) cells in small (˜100 μm) alginate based microcapsules.

It is expected that the amount of intracellular cryoprotectant required for vitrifying the ES cells can be lowered to a low, nontoxic level (i.e., ≦1.5 M) by combining the quartz microcapillary technique and the alginate-based cell microencapsulation technique. One challenge for the quartz microcapillary technique is that like all open system, it is vulnerable to potential contamination. Microencapsulation of the ES cells in alginate matrix should reduce the probability of contamination because the low permeability of the alginate matrix should keep any contaminants away from the cells encapsulated in the matrix. This challenge can be further overcome by adding two additional columns of the vitrification solution without cells (i.e., one above and one below the vitrification solution column loaded with microencapsulated cells with an air gap between any two adjacent columns) to prevent the microencapsulated cells from directly contacting liquid nitrogen. This system actually resembles the closed pulled straw system reported in the literature.

### Example II

The morphological changes of small (˜100 μm) alginate microcapsules and the biophysical alterations of water in the microcapsules during cryopreservation with a cooling rate of 100° C./min were studied using cryomicroscopy and scanning calorimetry. It was found that water in the small microcapsules can be preferentially vitrified over water in the bulk solution in the presence of 10% (v/v) or more dimethylsulfoxide (a cryoprotectant), which resulted in an intact morphology of the microcapsules post cryopreservation. A small amount of Ca2+(up to 0.15 M) was also found to help maintain the microcapsule integrity during cryopreservation, which is attributed to the enhancement of the alginate matrix strength by Ca2+rather than promoting vitrification of water in the microcapsules. The capability of the small alginate microcapsule to retain its intact morphology and augment vitrification of water in its space at a low concentration of cryoprotectants (i.e., 10% (v/v)) and an easily achievable fast cooling rate (i.e., 100° C./min), make it a great system to protect the encapsulated living cells that are sensitive to ice formation and high concentration of cryoprotectants from injury during cryopreservation.

In the present example, the morphological and biophysical observations obtained using cryomicroscopy and scanning calorimetry in cryopreservation of the small alginate microcapsules are described. The observations were further compared and analyzed to understand the fate of water in the microcapsules during cryopreservation. It was found that the intact morphology of the small alginate microcapsules could be well retained post cryopreservation and more importantly, water in the microcapsules can be preferentially vitrified at a low concentration of cryoprotectants and easily achievable fast cooling rates. Therefore, the small alginate microcapsule is a great system for not only encapsulating sensitive (to ice formation and high concentration of cryoprotectants) living cells but protecting the cells from cryoinjury during cryopreservation.

Small (˜100 μm or less) alginate microcapsules were prepared using the electrostatic spray method by extruding an aqueous solution of 2% (w/v) purified sodium alginate (type M, Medipol, Lausanne, Switzerland) at 3.5 ml/h through a blunt syringe needle (150 μm in ID and 300 μm in OD) into 0.15 M calcium chloride gellation solution as detailed elsewhere 14. An electrostatic voltage (16.5 kV) was applied between the needle tip and the free surface of the gellation solution at a distance of 5.5 mm. The microcapsules formed in the gellation solution were collected by centrifuging at 200g for 3 min and re-suspended in normal physiologic (1×) saline with 0-15% (v/v) dimethylsulfoxide (DMSO, Sigma, St Louis, Mo.) and/or 0-0.15 M calcium chloride for further cryomicroscopic and calorimetric studies.

Cryomicroscopic studies were performed using a Linkam (Waterfield, UK) FDCS196 freeze drying stage mounted on an Olympus BX 51 microscope. The microscope is equipped with a Qlmaging (Surrey, BC, Canada) QICAM CCD microscope camera for digital imaging. For each experiment, a total of 5 μl sample solution (sandwiched between the bottom surface of the quartz sample crucible for the FDCS196 stage and a 9 mm round coverglass) was cooled at 10° C./min from room temperature to −4° C., held for 3 min, cooled at 100° C./min to −80° C., held for 5 min, and heated to 25° C. at 100° C./min.

For calorimetric studies using a Linkam DSC600 stage, a total of 3 μl sample solutions (sandwiched between the bottom surface of the sapphire sample crucible for the FDCS196 stage and a 3 mm round coverglass) was used. The cryopreservation protocol was the same as that used for cryomicroscopic studies except that the samples were heated from −80° C. to 25° C. at 5° C./min in order to clearly record the heat flux during heating. The heat flux vs. temperature curve of a background scan for the empty crucible with a coverglass was then subtracted from that of the sample loaded crucible to give the net heat flux as a result of heating the sample. The peak area on the net heat flux vs. temperature curve was calculated using the peak analysis tool (assuming a linear baseline) in the Linkam Linksys32 software designed for the DSC600 stage.

FIG. 10 shows the morphology of microcapsules in 1× saline before cryopreservation (FIG. 10A) and post cryopreservation with DMSO of different concentrations. Without DMSO, the microcapsules become wrinkled (FIG. 10B) post cryopreservation. Less damage is observable (FIG. 10C) in the presence of 5% (v/v) DMSO and the microcapsules appear intact (FIG. 10D) post cryopreservation with 10% (v/v) DMSO. The rough appearance of the microcapsules in FIGS. 10B&C presumably is due to the formation of ice in the microcapsules 15 that mechanically disrupted the alginate matrix during cryopreservation. The intact morphology of microcapsules shown in FIG. 10D suggests that ice formation in the microcapsules is negligible during cryopreservation in the presence of 10% (v/v).

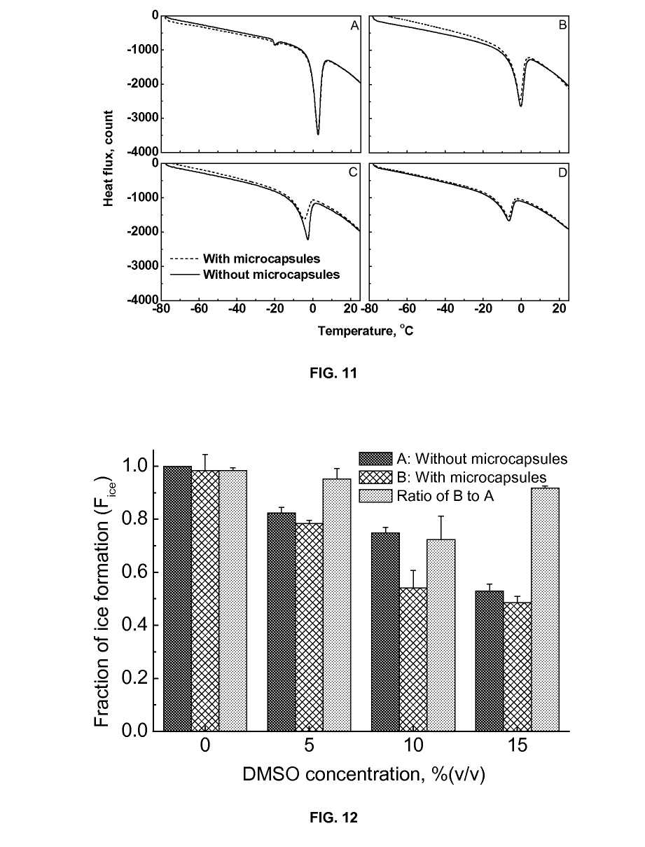

The above speculations of the fate of water in the microcapsules during cryopreservation can be verified by data obtained from calorimetric studies shown in FIG. 11, where the net heat flux as a function of temperature for samples (1× saline) with 0, 5, 10, and 15% (v/v) DMSO is given in panels A, B, C, and D, respectively. Being consistent with the literature, a minor peak (at ˜−21° C.) due to eutectic melting is observable for samples without DMSO (FIG. 11A) and the peak disappears in the presence of DMSO (FIG. 11B-D). The major peak in each panel of the figure is due to melting of ice and its area is proportional to the amount of ice formed in a sample during cooling. Clearly, without DMSO, the peak areas of samples with and without microcapsules are similar (FIG. 11A), indicating the fate of water in the microcapsules is not different from that in the bulk solution. The peak area of samples with DMSO is always smaller than that without DMSO and the area decreases monotonically with the increase of DMSO from 5-15% (v/v) (FIG. 11B-D). The reduction in peak area with the increase of DMSO could be due to two reasons: the decrease of total saline (or water) in the sample and less extent of ice formation in the sample during cooling. In order to compare the extent of ice formation in samples with different amount of DMSO, the fraction of ice formation (F_{ice}) in a sample was quantified as the ratio of the peak area of the sample to that of available saline in the sample as follows:

$${F}_{ice} = {PA}_{sample} / ( \frac{{m}_{saline}}{{m}_{sample}} {PA}_{saline} ) \qquad ( 1 )$$

where PAₛₐₘₚₗₑ represents the peak area of a sample shown in FIG. 11 A-D, PAₛₐₗᵢₙₑ represents the peak area of the pure saline sample without microcapsules shown in FIG. 11A, and mₛₐₘₚₗₑ and mₛₐₗᵢₙₑ represents the total weight of a sample and the weight of saline in the sample, respectively. F_{ice} together with the ratio of F_{ice} in samples with microcapsules to F_{ice} in samples without microcapsules at various DMSO concentrations is shown FIG. 12. Clearly, F_{ice} decreases with the increase of DMSO in all samples, which is consistent with the literature. F_{ice} in samples with microcapsules are only slightly less than that in samples without microcapsules when DMSO concentration is less than 10% (v/v) as indicated by their ratio (close to 1) in the figure. When the DMSO concentration is 10% (v/v), however, F_{ice} in samples with microcapsules is much less than that in samples without microcapsules. Interestingly, the difference in F_{ice} between samples with and without microcapsule decreases again when DMSO concentration is increased to 15% (v/v). Moreover, F_{ice} in the samples with microcapsules in the presence of 10 and 15% (v/v) DMSO are not significantly different. These observations indicate that with 10% (v/v) DMSO (or more), water encapsulated in the alginate microcapsule become preferentially vitrified over water in the bulk solution. In other words, encapsulation of water in the small alginate microcapsules can significantly augment its vitrification only in the presence of sufficient cryoprotectants (e.g., 10% (v/v) DMSO or higher). By putting together the cryomicroscopic and calorimetric data (i.e., FIGS. 10 and 11 vs. FIG. 12), it can be concluded that water in the microcapsules was vitrified in the presence of 10% (v/v, or higher) DMSO, which resulted in nearly intact microcapsules post cryopreservation at a dangerous cooling rate of 100° C./min that is well-recognized in cryobiology to prevent dehydration and promote ice formation in cells suspended in bulk solutions during cryopreservation. Since mammalian cells are generally smaller than the alginate microcapsules and water in a smaller confinement is generally easier to be vitrified, it is expected that as with the microcapsules, cells encapsulated in the small microcapsules can be vitrified under the cryopreservation protocol with 100° C./min cooling rate and 10% (v/v) DMSO. Therefore, microencapsulation of sensitive (to ice formation and high concentration of cryoprotectants) living cells in the small alginate microcapsules can provide a novel approach to achieve cryopreservation of the cells by ‘semi-vitrification’ (i.e., ice forms outside the microcapsules only and has no direct contact with the cells) at a cryoprotectant (DMSO) concentration as low as 10% (˜1.5 M) using an easily achievable cooling rate (e.g., 100° C./min or higher). This new approach can potentially avoid the technical challenge associated with creating an ultrafast cooling rate (>200,000° C./min) to vitrify living cells suspended in bulk solution at a low concentration of cryoprotectant (<2 M) and the toxicity problem of extremely high concentration of cryoprotectants (>4 M) required for vitrifying cells suspended in bulk solution at an easily achievable cooling rate (e.g., 100-1000° C./min).

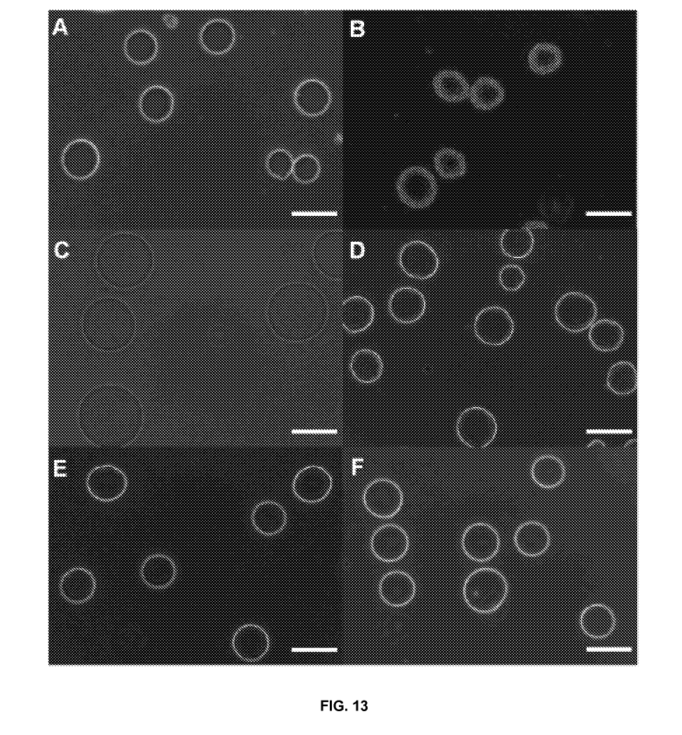

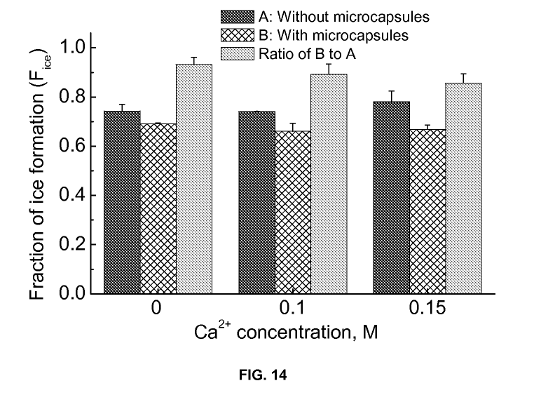

Since the alginate matrix in the small alginate microcapsules is maintained by bridging calcium cations (Ca2+), understanding the effect of Ca2+ on the microcapsule morphology and the fate of water in the microcapsule is of great interest. FIG. 4a shows that microcapsules in 1× saline with 0.15 M Ca2+ are smaller than that in 1× saline without Ca2+ (˜80 μm in FIG. 13A vs. 100 μm in FIG. 10A), indicating the alginate matrix become tighter with the increase of Ca2+ concentration. The morphology of the microcapsule post cryopreservation in the presence of 0.15 M Ca2+ without DMSO appears as wrinkled pattern of concentric rings, particularly on the peripheral (FIG. 13B). FIG. 4a C shows the morphology of microcapsules post cryopreservation in 1× saline with 7.5% DMSO in the absence of Ca2+. Although the surface appears smoother than that of microcapsules post cryopreservation with a less amount of DMSO (FIGS. 10B&C), the microcapsules are swollen and their boundaries become blurry (FIG. 13C). In the presence of 0.05 M Ca2+ and 7.5% DMSO, however, the morphology of the microcapsules post cryopreservation appears nearly intact with minor damages observable only on the edge of the microcapsules (FIG. 13D). The microcapsules retain intact morphology post cryopreservation if the Ca2+ concentration is increased to 0.01 M or higher in the presence of 7.5% DMSO (FIGS. 13D&E). FIG. 14 shows the F_{ice} in samples with 7.5% (v/v) DMSO and 0-0.15 M Ca2+. This figure shows that F_{ice} in samples with and without microcapsules and their ratios are not significantly different. Therefore, it appears that Ca2+ doesn't significantly affect the fate of water encapsulated in the alginate microcapsules although it can help maintain the microcapsule integrity at a sub-optimal DMSO concentration (i.e., 7.5% (v/v). These observations suggest that Ca2+ helps maintain the microcapsule morphology probably by strengthening the alginate matrix to resist irreversible damage as a result of ice formation rather than by preventing ice formation in the microcapsules.

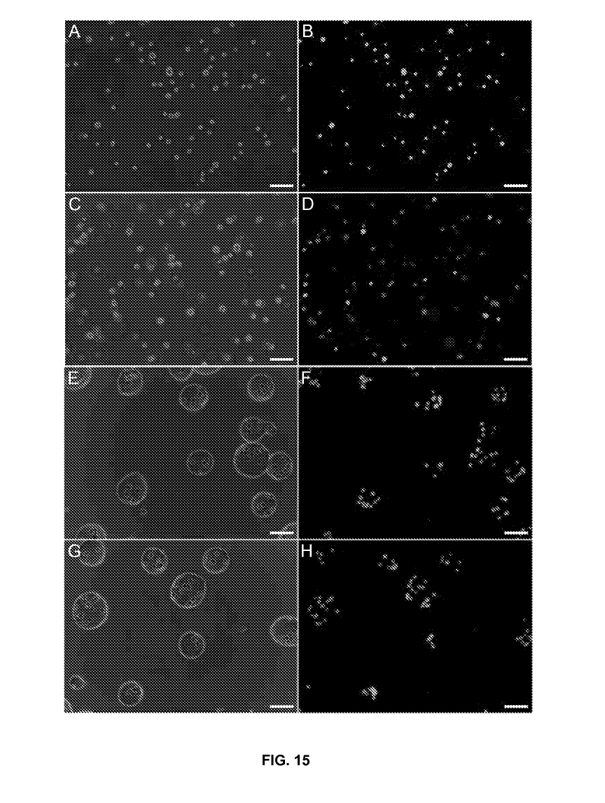

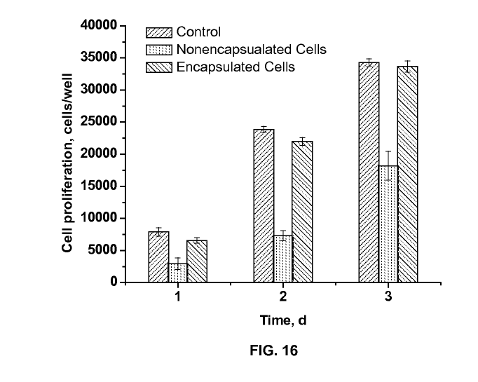

In summary, water in small (˜100 μm) alginate microcapsules can be preferentially vitrified resulting in intact microcapsules post cryopreservation in the presence of 10% (v/v) or more DMSO. Ca2+ could help maintain microcapsule integrity post cryopreservation by enhancing the strength of the alginate matrix rather than promoting vitrification of water in the microcapsules. The small alginate microcapsules could be very useful for cryopreservation of important living cells that are sensitive to both freezing (ice formation) and high concentrations of cryoprotectants. FIG. 15 illustrates typical phase (A, C, E, and G) and fluorescent (B, D, F, and H) micrographs of control (non-microencapsulated, A-D) and microencapsulated (E-H)C3H10T1/2 mouse mesenchymal stem cells cells and FIG. 16 illustrates proliferation of the encapsulated and non-microencapsulated C3H10T1/2 mouse mesenchymal stem cells after vitrification shown in FIG. 15 together with control (fresh without vitrification) 3 days after seeding them in 96 well plate and culturing in normal medium at 37° C. and 5% CO₂.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed:

1. A method for cryopreserving a cell comprising:
encapsulating a cell in a microcapsule, the microcapsule having a diameter of less than about 100 μm;
vitrifying the encapsulated cell in a vitrifying solution comprising a cryoprotectant, wherein the cell is cooled at a rate of equal to or greater than 30,000° C./min and the cryoprotectant is present at a concentration of less than or equal to 1.5 M.

2. The method of claim 1, wherein the cell is a mammalian cell.

3. The method of claim 1, wherein the microcapsule comprises a biocompatible material.

4. The method of claim 1, wherein the microcapsule comprises alginate.

5. The method of claim 1, wherein said cell is an oocyte, a sperm, a stem cell, an embryo, or a zygote.

6. The method of claim 1, wherein the encapsulated cell is vitrified in a capillary tube.

7. The method of claim 6, wherein said capillary tube comprises a material selected from the group consisting of plastic, glass, quartz, stainless steel, sapphire, silver, copper, diamond, gold, titanium, palladium, platinum.

8. The method of claim 7, wherein said material is quartz.

9. The method of claim 1, wherein said cell is cooled at a rate equal to or greater than 50,000° C./min.

10. The method of claim 1, wherein said cell is cooled at a rate equal to or greater than 100,000° C./min.

11. The method of claim 1, wherein the cryoprotectant is selected from one or more of the group consisting of a sugar, glycerol, ethylene glycol, 1,2-propanediol, and DMSO.

12. The method of claim 1, wherein the cryoprotectant comprises 1,2-propanediol.

13. The method of claim 1, wherein vitrification of the encapsulated cell occurs in the absence of ice formation.

14. The method of claim 1, wherein the vitrification solution further comprises at least one nanoparticle or microparticle.

15. The method of claim 14, wherein the nanoparticle or microparticle comprises carbon or a noble metal.

16. The method of claim 14, wherein the nanoparticle or microparticle is selected from the group consisting of gold, silver, titanium, palladium, platinum, and copper.

17. The method of claim 1, wherein the vitrification solution further comprises polymers or peptides that inhibit ice nucleation in the vitrification solution.

18. The method of claim 17, wherein the polymer or peptide is selected from the group consisting of polyvinyl alcohol, polyglycerol, and antifreeze proteins.

19. The method of claim 1, wherein the cryoprotectant is present at a concentration of less than or equal to 1.0 M.

20. The method of claim 1, wherein the cryoprotectant is present at a concentration of less than or equal to 0.5 M.
